(19) United States
(12) Patent Application Publication
(10) Pub. No.: US 2008/0125752 A1
(43) Pub. Date: May 29, 2008

(54) Catheter assembly having a modified reinforcement layer

(75) Inventors: Richard C. Gunderson, Maple Grove, MN (US); Anthony F. Tassoni, Ramsey, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: Boston Scientific Scimed, Inc.

(21) Appl. No.: 11/501,287

(22) Filed: Aug. 9, 2006

(51) Int. Cl. A61M 25/16; B23P 11/00

(52) U.S. Cl. 604/527; 604/524; 29/505

(57) ABSTRACT

Catheter shafts and methods for making and using the same. An example catheter shaft includes an elongate liner, a reinforcement member disposed on the liner, and a sheath disposed on the reinforcement member. The reinforcement member may include one or more circumferential and/or spot welds.

### FIELD OF THE INVENTION

The present invention pertains to intra-lumenal medical devices. More particularly, the present invention pertains to catheters and catheter shafts with a modified reinforcement layer.

### BACKGROUND

A wide variety of intra-lumenal medical devices have been developed for medical use, for example, intravascular use. Some of these devices include catheters and catheter shafts with a reinforcement layer. These catheter shafts are manufactured by any one of a variety of different manufacturing methods. Of the known catheters, catheter shafts and manufacturing methods, each has certain advantages and disadvantages. There is an ongoing need to provide catheters and catheter shafts as well as methods for making and using catheters and catheter shafts.

### BRIEF SUMMARY

The invention provides design, material and manufacturing method alternatives for medical devices, for example, catheters and catheter shafts. Exemplary catheter shafts include an elongate liner, a reinforcement layer or member disposed on the liner, and a sheath disposed on the reinforcement layer. The reinforcement layer may include a braid with one or more circumferential and/or spot welds.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a plan view of an example catheter disposed in a body lumen;

FIG. 2 is a partially cutaway view of an example catheter shaft;

FIG. 3 is a close up illustration of a portion of the catheter shaft shown in FIG. 2;

FIG. 4 depicts an example welding method that may be utilized during the manufacturing of an example catheter shaft;

FIG. 5 depicts another example welding method that may be utilized during the manufacturing of an example catheter shaft;

FIG. 6 is a side view of another example catheter shaft;

FIG. 7 is a side view of an example crimp member;

FIG. 8 is a side view of another example crimp member;

FIG. 9 is a side view depicting an example manufacturing method for a catheter shaft;

FIG. 10 is a side view depicting a example manufacturing method for a catheter shaft; and

FIG. 11 is a side view depicting an example manufacturing method for a catheter shaft.

### DETAILED DESCRIPTION

The following description should be read with reference to the drawings wherein like reference numerals indicate like elements throughout the several views. The detailed description and drawings illustrate example embodiments of the claimed invention.

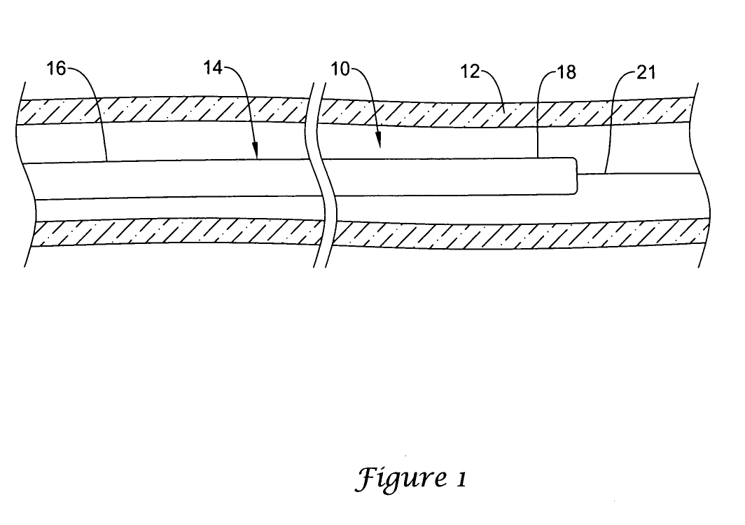

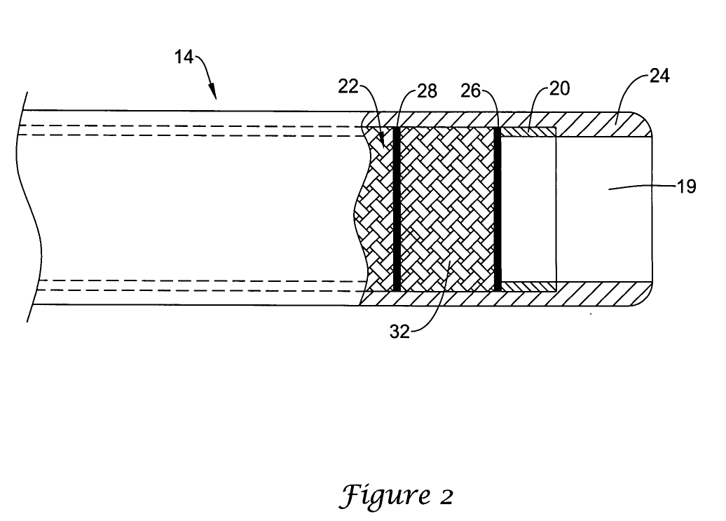

FIG. 1 is a plan view of an example catheter 10 disposed in a blood vessel 12. Catheter 10 may include a catheter shaft 14 having a proximal region 16 that typically extends outside of the patient's body when catheter 10 is in use, a distal region 18, and a lumen 19 (not shown, best seen in FIG. 2) extending at least partially therebetween. Disposing catheter 10 in blood vessel 12 may include advancing catheter 10 over a guidewire 21. Catheter 10 may be used for intravascular procedures according to common practice and procedure. For example, catheter 10 may be a diagnostic, therapeutic, or guide catheter used in conjunction with other medical devices such as guidewires and other catheters. Of course, numerous other uses are known amongst clinicians for catheters and other similarly configured medical devices.

A number of catheter shafts include a reinforcing structure such as a braid. Braids are made up of a number of individual wires or wire filaments that are woven together in the form of a braid. It can be readily appreciated that at some point along the catheter, the braid has a distal end, likely corresponding to the distal end of the catheter, that may include a number of loose ends of the wires making up the braid. The distal end of the catheter may not incorporate a design feature for controlling the ends of the braid. This may increase the chances of the braid wires elongating beyond an inner liner during manufacturing, protruding from the catheter post manufacturing, moving during or after manufacturing, or otherwise disrupting the overall function of the catheter. The present invention incorporates design and manufacturing method innovations that address this and other issues.

FIG. 2 depicts a side view of a portion of catheter shaft 14. Shaft 14 includes an elongate tubular liner 20, a reinforcement layer or member 22 disposed on the liner, and a sheath 24 disposed on reinforcement layer 22. Liner 20 may include a polymer such as polytetrafluoroethylene (PTFE) or any other suitable lubricious material. Some examples of other suitable polymers may include ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVC), polycarbonates, ionomers, biocompatible polymers, other suitable materials, or mixtures, combinations, copolymers thereof, polymer/metal composites, and the like. It should be noted that liner 20 may include any other appropriate material including any other suitable material listed herein. Sheath 24 may also include a polymer, for example, including any of the polymers listed above.

Reinforcement layer 22, which is disposed on liner 20, may have any one of a number of different forms. For example, reinforcement layer 22 may be a braid as seen in FIG. 2. According to this embodiment, one or more wires or wire filaments may be woven together into a braid. The wires may vary in size (e.g., thickness) and shape so as to include round wire, flat wires or wires of any other shape. Alternatively, reinforcement layer 22 may be a coil, a mesh, a matrix, and the like, or have any other suitable configuration.

Regardless of what form reinforcement layer 22 is in, a number of different materials may be used to make it. For example, reinforcement layer 22 may be a metal, metal alloy, polymer such as KEVLAR® (see the examples above), metal/polymer composite, and the like, or any other suitable material. Some examples of suitable metals and metal alloys include stainless steel, such as 304V, 304L, and 316LV stainless steel; mild steel; nickel-titanium alloy such as linear-elastic or super-elastic nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), hastelloy, monel 400, inconel 825, or the like; other Co—Cr alloys; platinum enriched stainless steel; or other suitable material.

In at least some embodiments, reinforcement member 22 includes a radiopaque material. Radiopaque materials are understood to be materials capable of producing a visible image on a fluoroscopy screen or another imaging technique during a medical procedure. This visible image aids the user of catheter 10 in determining its location. Some examples of radiopaque materials can include, but are not limited to, gold, platinum, molybdenum, palladium, tantalum, iridium, tungsten or tungsten alloy, plastic material loaded with a radiopaque filler, and the like.

Reinforcement member 22 may include one or more welds, for example, a first weld 26 and a second weld 28. Welds 26/28 generally span the circumference of reinforcement member 22. Circumferential welds 26/28 thus form weld bands or rings that circumscribe or otherwise go all the way around reinforcement member 22. Although being described as circumferential, welds 26/28 are not intended to be limited to being only circular in shape, as a number of variations are contemplated. For example, welds 26/28 can be oval, polygonal, helical, irregular, and the like, or any other suitable shape.

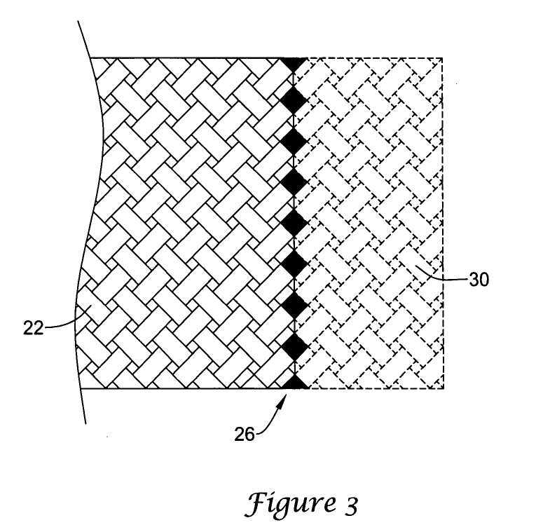

First weld 26 is typically disposed near the distal end of reinforcement member 22. In at least some embodiments, reinforcement member 22 is a braid. As more clearly seen in FIG. 3, weld 26 joins together intersecting or adjoining wire filaments that form braid 22. This forms the ring-like weld 26 disposed along the circumference of braid 22 that attaches together the wires of braid 22. With weld 26 forming a circumferential line where the wires of braid 22 are held together, an excess portion 30 of braid 22 can be trimmed away, as depicted in FIG. 3 in phantom line, so that weld 26 becomes the distal end of reinforcement member 22.

It can be appreciated that with weld 26 disposed at the distal end of braid 22, essentially all potential “loose ends” of the wires making up braid 22 are held together. This helps prevent the ends of the wires making up braid 22 from fraying, losing and/or changing position, protruding, etc. In addition, weld 26 may be more easily visualized using fluoroscopic imaging than a plurality of loose wire ends. Thus, the presence of weld 26 helps to improve the overall integrity of reinforcement member 22, as well as catheter 10, and can provide a number of desirable properties that can improve the intervention for which catheter 10 is intended to be used for.

Referring now back to FIG. 2, second weld 28, which generally takes the same circumferential form as first weld 26, is typically disposed a distance proximal of first weld 26. The amount of spacing can vary in a number of the various contemplated embodiments. For example, the welds 26/28 can be spaced about 1-50 millimeters or more. However, any suitable length can be utilized. In addition, one or more additional welds can also be placed along the length of reinforcement member 22 and spaced a similar distance from first weld 26 and/or second weld 28.

The space between first weld 26 and second weld 28 defines a crimpable region 32 of reinforcement member 22. In at least some embodiments, crimpable region 32 is annealed. Being annealed is understood to mean that crimpable region 32 is heated and then slowly cooled in order to allow the reinforcement member in that region to be deformed (maintain the shape when crimped). Crimpable region 32, by virtue of being annealed, allows crimpable region 32 to be crimped or otherwise secured onto liner 20. This may improve the attachment of reinforcement member 22 with liner 20.

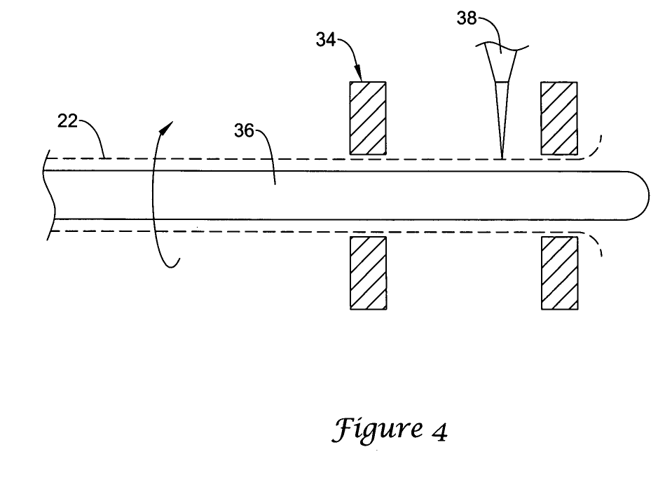

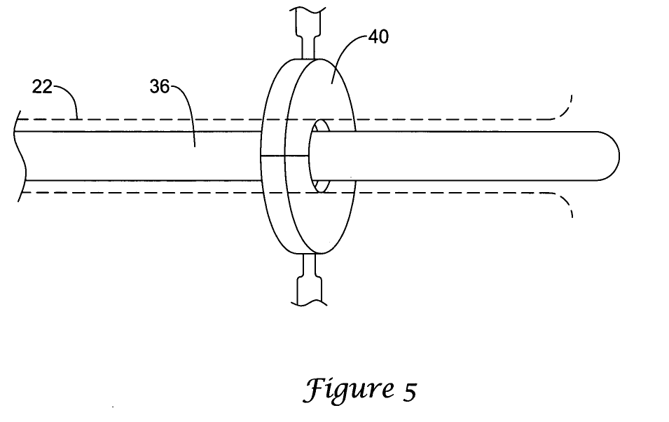

A number of different welding methods can be utilized to define welds 26/28. For example, welds 26/28 can be defined using laser spot welding apparatus 34 as depicted in FIG. 4. According to this embodiment, reinforcement member 22 can be disposed on a rotatable mandrel 36 and positioned adjacent a laser source 38. When the laser source 38 is activated, mandrel 36 and reinforcement member 22 can be rotated so that the laser defines a circumferential weld, for example weld 26. Another weld, for example weld 28, can be defined by longitudinally shifting mandrel 36 and the activating laser source 38. In some other embodiments, welds 26/28 can be formed using a compressed resistance welding technique as illustrated in FIG. 5. According to this embodiment, for example, a clamp-like electrode 40 can be disposed on reinforcement member 22 and activated to form welds 26/28. It can be appreciated that any other suitable welding technique (e.g., induction welding, TIG welding, microplasma welding, electron beam, friction or inertia welding, and the like) can be utilized without departing from the spirit of the invention.

With the aforementioned structures and methods in mind, a number of manufacturing strategies can be utilized to produce catheter 10. One aspect of the manufacturing method may be directed toward the manufacturing of reinforcing member 22. This may include, in embodiments where reinforcing member 22 is a braid, winding a wire onto a core to form a braid. The braid can be cut to a suitable length and put on a welding mandrel, for example mandrel 36. Any of the welding techniques described above can be used to form welds 26/28 on reinforcing member 22. The excess portion 30 can be trimmed or ground from reinforcing member 22. The trimmed reinforcement member 22 can be annealed to define crimpable region 32.

Another aspect of the manufacturing method includes incorporating the welded and annealed reinforcement member 22 into catheter 10. For example, liner 20 can be disposed on a mandrel. Reinforcement member 22 can be disposed on liner 20. With reinforcement member 22 positioned, crimpable region 32 can be crimped onto liner 20. Sheath 24 can be disposed on reinforcement member 22. A heat shrink tube can be disposed on sheath 24, and catheter 10 can be heated so that liner 20, reinforcement member 22, and sheath 24 are bonded by heat. The heat shrink tube can then be removed and the ends of catheter 10 can be trimmed, if desired, to meet the desired specifications. Additional optional steps may also be performed, such as molding a distal tip and/or a proximal hub onto catheter 10.

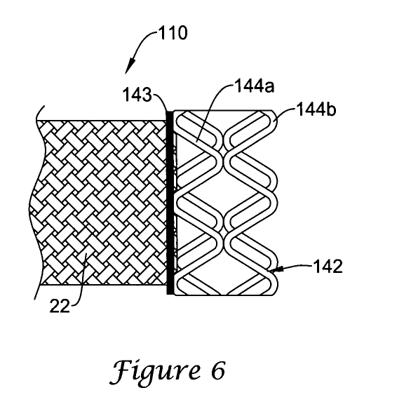

FIG. 6 illustrates a portion of another example catheter 110 that is similar in form and function to catheter 10. Catheter 110 include a crimpable ring 142 coupled to reinforcement member 22, for example, by welding. Welding crimpable ring 142 to reinforcement member 22 may include a circumferential or spot weld 143 that attaches these structures. In some embodiments, crimpable ring 142 is disposed over a portion of reinforcement member 22 and welded thereto. According to this embodiment, the inside surface of ring is attached to the outside surface of reinforcement member 22. Alternatively, ring 142 may be welded along the inside surface of reinforcement member 22 or ring 142 may abut the end of reinforcement member 22.

As the name suggests, crimpable ring 142 defines a crimpable region (much like crimpable region 32 of catheter 10) that not only secures together the ends of reinforcement member 22 but also aids in the attachment of reinforcement member 22 to liner 20. This is because crimpable ring 142 can be crimped onto liner 20 analogously to how crimp zone 32 is crimped.

The exact form of crimpable ring 142 can vary significantly. For example, any suitable material can be utilized to make ring 142 including those metals disclosed herein. In some embodiments, ring 142 is made from the same material as reinforcement member 22. Alternatively, differing material can be utilized. In some embodiments, crimpable ring 142 can be made from a radiopaque material to aid in visualization of catheter 110.

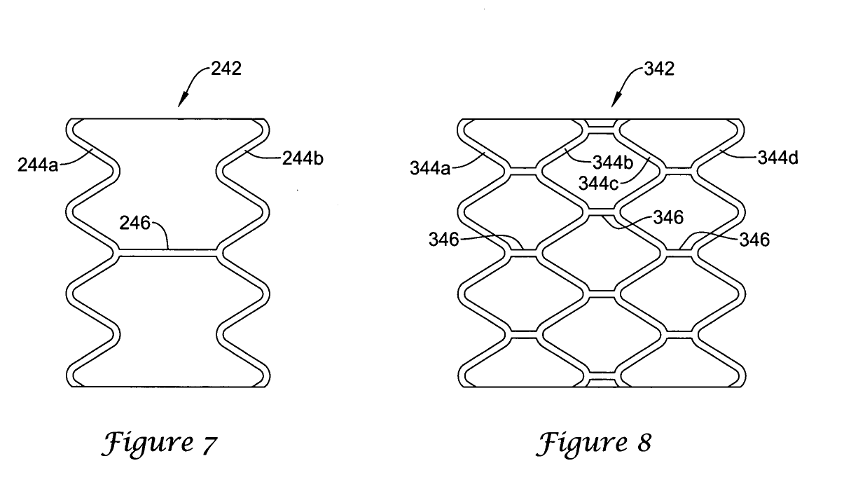

The shape and configuration of shapeable ring 142 can also vary. For example, FIG. 6 depicts ring 142 as being generally cylindrical in shape and being made up of a pair of parallel wires 144a/144b that “zigzag” and are attached at opposing peaks of the zigzag. Numerous other arrangements are contemplated. For example, FIG. 7 illustrates another example crimpable ring 242 that similarly includes a pair of parallel zigzag wires 244a/244b. Unlike wires 144a/144b, wires 244a/244b are attached by a longitudinal rib 246. In addition, FIG. 8 illustrates another example crimpable ring 342 that demonstrates that essentially any number of parallel wires can be utilized such as four wires 344a/344b/344c/344d (as well as three wires, five wires, six wires, or more). Wires 344a/344b/344c/344d may be attached together like wires 114a/114b of ring 142, with one or more longitudinal ribs 346, or in any other suitable manner.

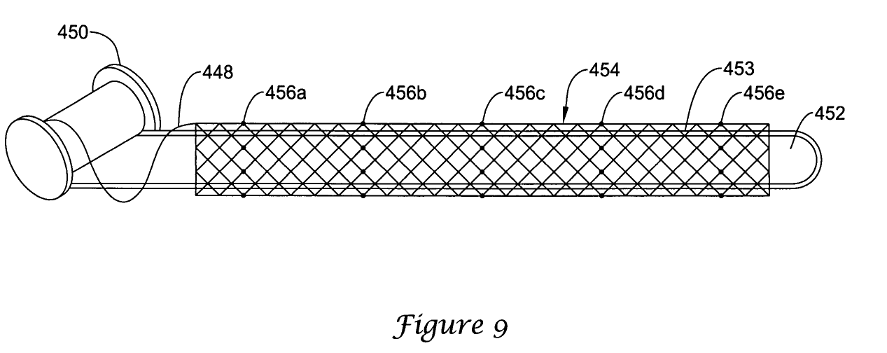

Another example method for manufacturing a catheter is depicted in FIGS. 9-11. Turning now to FIG. 9, wire 448 on a spool 450 can be wound about a mandrel 452 (which can have a liner 453 similar to liner 20 disposed thereon) into the form of a braid 454 that can be similar in form and function to reinforcement member 22. A series of circumferential spot welds 456a/456b/456c/456d/456e can be defined at a plurality of longitudinal locations along braid 454. Welds 456a/456b/456c/456d/456e, like welds 26/28, can circumscribe braid 454. The number and arrangement of welds 456a/456b/456c/456d/456e can vary. For example, one, two, three, four, five, six, seven, eight, or more welds can be utilized. These welds can be arranged regularly (i.e., equally spaced), irregularly, become increasingly closer together or further apart, or otherwise be arranged in any suitable way. In some embodiment, welds 456a/456b/456c/456d/456e are spaced about 1-75 inches apart.

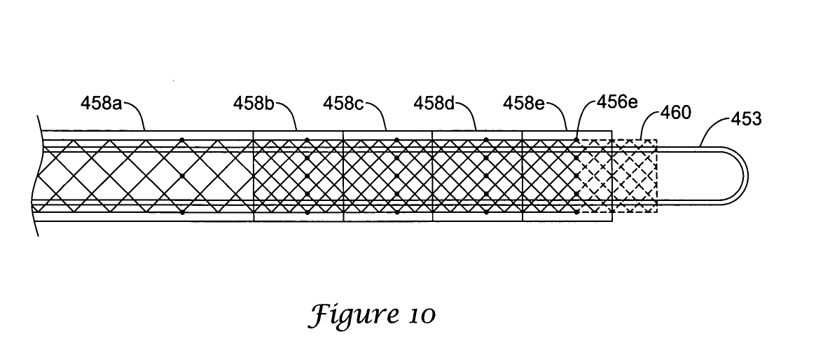

With welds 456a/456b/456c/456d/456e defined in braid 454, one or more polymeric members 458a/458b/458c/458d/458e can be disposed over braid 454 as shown in FIG. 10. Polymeric members 458a/458b/458c/458d/458e may be the same in material composition or may differ. In some embodiments, polymeric members 458a/458b/458c/458d/458e may differ in durometer, for example, becoming more flexible in one (e.g., distal) direction. The assembly depicted in FIG. 10 can be heated so that polymeric members 458a/458b/458c/458d/458e and/or liner 453 reflow and secure with braid 454. The distal section 460 of braid 454 can be trimmed up to, for example, weld 456e.

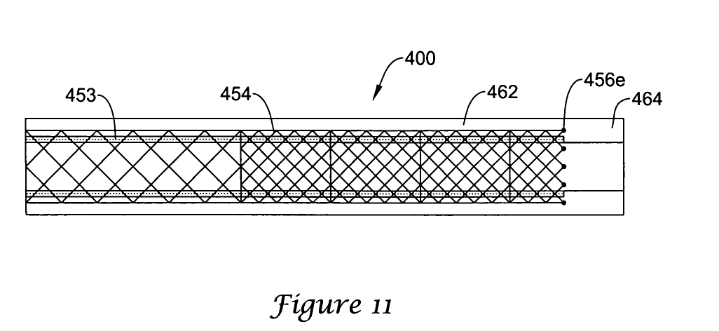

Turning now to FIG. 11, following the heating step, the outer surface 462 may be defined in the finished catheter 400 by the series of polymeric members 458a/458b/458c/458d/458e or by the addition of another polymeric sheath. In some embodiments, a distal tip 464 is defined adjacent weld 456e. Tip 464 may be made up of the same or different materials as polymeric members 458a/458b/458c/458d/458e and/or outer surface 462.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A catheter shaft, comprising:
an elongate tubular liner;
a reinforcement layer disposed on the liner, the reinforcement layer having a proximal region and a distal region;
wherein the reinforcement layer includes a first weld disposed adjacent the distal region, the first weld circumscribing the reinforcement layer, and a second weld disposed proximal of the first weld, the second weld circumscribing the reinforcement layer; and
a sheath disposed on the reinforcement layer.

2. The catheter shaft of claim 1, wherein the liner includes polytetrafluoroethylene.

3. The catheter shaft of claim 1, wherein the reinforcement layer includes a metal.

4. The catheter shaft of claim 3, wherein the metal includes stainless steel.

5. The catheter shaft of claim 1, wherein the reinforcement layer includes a braid.

6. The catheter shaft of claim 1, wherein a crimp region is defined between the first weld and the second weld.

7. The catheter shaft of claim 6, wherein at least a portion of the reinforcement member in the crimp region is annealed.

8. The catheter shaft of claim 7, wherein the crimp region is crimped onto the liner.

9. The catheter shaft of claim 1, wherein the first weld is disposed at a distal end of the reinforcement layer.

10. A catheter shaft, comprising:
an elongate tubular liner;
a reinforcement braid disposed on the liner, the reinforcement braid having a proximal region and a distal region;
wherein the reinforcement braid includes a plurality of wires braided together;
a first weld attaching one or more of the wires together at a first longitudinal position;
a second weld attaching one or more of the wires together at a second longitudinal position;
wherein the first position and the second position are disposed adjacent the distal region of the reinforcement braid; and
a sheath disposed on the reinforcement braid.

11. The catheter shaft of claim 10, wherein the reinforcement braid has a circumference, and wherein the first weld and the second weld are both disposed within a circumferential weld that spans the circumference.

12. The catheter shaft of claim 10, wherein the reinforcement braid has a circumference and a longitudinal axis, wherein the first weld is a first circumferential weld that spans the circumference and the second weld is a second circumferential weld that spans the circumference, and wherein the first position and the second position are spaced from one another along the longitudinal axis.

13. A catheter shaft, comprising:
an elongate tubular liner;
a reinforcement braid disposed on the liner, the braid having a proximal region and a distal region;
a crimpable member welded to the distal region of the braid; and
a sheath disposed on the braid.

14. A method for manufacturing a catheter shaft, comprising the steps of:
providing a reinforcement member;
welding a first portion of the reinforcing member;
welding a second portion of the reinforcing member;
annealing the reinforcing member;
providing an elongate tubular liner;
disposing the reinforcing member on the liner; and
disposing a sheath on the reinforcing member.

15. The method of claim 14, wherein the step of welding a first portion of the reinforcing member, the step of welding a second portion of the reinforcing member, or both includes welding a circumferential weld around the reinforcing member.

16. The method of claim 14, wherein the step of welding a first portion of the reinforcing member, the step of welding a second portion of the reinforcing member, or both includes spot welding the reinforcing member.

17. The method of claim 14, wherein the welding steps define a crimp region in the reinforcing member and wherein the step of annealing the reinforcing member includes annealing the crimp region.

18. The method of claim 17, wherein the step of disposing the reinforcing member on the liner includes crimping the crimp region onto the liner.

19. The method of claim 14, wherein the reinforcement member includes a distal end and wherein the step of welding a first portion of the reinforcing member includes welding the reinforcement member at a location proximally of the distal end.

20. The method of claim 19, further comprising the step of trimming of an excess section of the reinforcement member from the weld at the first portion to the distal end.

21. A method for manufacturing a catheter shaft, comprising the steps of:
providing an elongate tubular braid, the braid having a proximal region and a distal region;
welding a first circumferential portion of the distal region of the braid;
welding a second circumferential portion of the distal region of the braid, the second circumferential portion being disposed proximally of the first circumferential portion;
wherein a crimp region is defined between the first circumferential portion and the second circumferential portion;
annealing at least a portion of the crimp region;
providing an elongate tubular liner;
disposing the braid on the liner;
crimping the crimp region onto the liner; and
disposing a sheath on the braid.

22. A method for manufacturing a catheter shaft, comprising the steps of:
providing an elongate tubular liner;
providing a reinforcement braid, the braid having a proximal region and a distal region;
disposing the braid on the liner;
welding a crimpable member to the distal region of the braid; and
disposing a sheath on the braid.
